(19) United States
(12) Patent Application Publication
(10) Pub. No.: US 2020/0352611 A1
(43) Pub. Date: Nov. 12, 2020

(54) INTERLOCKING IMPLANT WITH EXPANDABLE FIXATION MEANS FOR L5-S1 SPINAL FUSION AND/OR FIXATION

(71) Applicant: NovApproach Spine, LLC, Alachua, FL (US)

(72) Inventor: Michael MacMillan, Gainesville, FL (US)

(21) Appl. No.: 16/868,488

(22) Filed: May 6, 2020

Related U.S. Application Data
(60) Provisional application No. 62/843,613, filed on May 6, 2019.

(51) Int. Cl. A61B 17/70

(52) U.S. Cl. CPC A61B 17/7055; A61B 2017/564

(57) ABSTRACT

The present invention provides for an improved implant, and surgical methods for installing same, that can securely fix the L5 vertebrae to the S1 sacrum within the disc space for patients exhibiting a wide range of anatomies. The present invention has at least one advantage of providing practical and advantageous spinal implants and implantation systems, methods and tools for accessing the spinal vertebrae to insert spinal implants in various manners that overcome the disadvantages of posterior and anterior lateral approaches thereto and minimize surgical trauma to the patient. Thus the present invention provides for the unmet need for an improved implant for performing spinal surgical procedures (e.g., spinal fusion and/or fixations) that can securely fix the L5 vertebrae to the S1 sacrum within the disc space for patients exhibiting a wide range of anatomies.

### CROSS-REFERENCE TO RELATED APPLICATIONS

This application claims the benefit of U.S. Provisional Application No. 62/843,613, having a filing date of May 6, 2019, the disclosure of which is hereby incorporated by reference in its entirety and all commonly owned.

### FIELD OF THE INVENTION

The present disclosure generally relates to implants for performing spinal surgical procedures in a human. More particularly, the disclosure generally relates to implants for performing spinal surgical procedures at the L5-S1 level.

### BACKGROUND OF THE INVENTION

Minimal invasive surgery (MIS) has several benefits over more traditional surgical techniques. Benefits include a lower rate of morbidity, reduced pain, reduced scarring, reduced blood loss and reduced recovery time and costs. In minimal invasive spine surgery, small incisions, typically about 0.5 to about 4.0 centimeters in length, are made in, for example a patient's back.

Spinal fusion and/or fixation within the intervertebral disc are performed for a number of pathological conditions. Restoring stability and eliminating abnormal motion can relieve pain. The surgical trauma necessary to access and prepare the fusion and/or fixation has been implicated as a source of patient discomfort. On each side of the body there is a continuous bony access corridor from the pelvis, through the sacrum, across the LS-S1 disc, and into the LS vertebral body. These corridors can be traversed by a tunnel that would not damage or endanger any sensitive visceral or neurologic structures. The tunnels could be accessed by percutaneous incisions in the gluteal area. Therefore, if a spinal fusion and/or fixation could be performed by using these tunnels patients could obtain the benefit of a spinal fusion and/or fixation without the potential harm created by an extensive surgical approach.

Because of the natural symmetry of the human body, the tunnels from the right and left sides tend to converge towards the same area within the LS-51 disc. There can occur a situation where the implants physically intersect each other, preventing one of the implants from crossing the midline and entering the LS vertebral body. It is ideal to have both implants end up in the LS vertebral body for maximal stabilization.

A wide variety of anterior, extraosseous fixation implants, primarily anterior plate systems, have also been proposed or surgically used. One type of anterior plate system involves a titanium plate with unicortical titanium bone screws that lock to the plate and are placed over the anterior surface of a vertebral body. Another type of anterior plate system involves the use of bicortical screws that do not lock to the plate. The bone screws have to be long enough to bite into both sides of the vertebral body to gain enough strength to obtain the needed stability. These devices are difficult to place due to the length of the screws, and damage occurs when the screws are placed improperly.

There have been several other implants introduced into the art to be used as spinal implants.

In a first example a spinal implant device for fixing or fusing the lumbosacral joint of a patient included endplates, structural support columns and fixation devices. However, this device did not provide an opening along the length of one or more fixators that affix both vertebrae simultaneously.

In a second example a spinal implant device for fixing or fusing the lumbosacral joint of a patient included screws that transfix L5 and S1 vertebra. However, the device did not provide an opening along the length of one or more fixators that affix both vertebrae simultaneously.

In a third example a spinal implant device for maintaining the motion at the lumbosacral joint of a patient included fixation devices and bearing surfaces. However, the device did not provide a means to affix both vertebrae simultaneously.

In a fourth example a spinal implant device for fixing or fusing the lumbosacral joint of a patient included a plurality of flanges distributed along the outer surface of the spinal implant body and can either be solid or with a hollow lumen along its length. However, the device did not provide an opening along its length that would enable a second spinal fixation implant to intersect its axis.

In a fifth example an intramedullary implant device for fixing or fusing long bone segments of a patient included a cannulated shaft of an implant body and a second implant device with a tang assembly. However, the device did not provide an opening along its length that would enable a second implant to intersect its axis at variable angles.

In a sixth example a spinal implant device for stabilizing facets of adjacent vertebrae in a patient included a body and shank and anchor portion along its length. However, the device did not provide an opening along its length that would enable a second spinal implant to intersect its axis at different positions.

In a seventh example a spinal implant device for fixing an intervertebral fusion and/or fixation cage in a disc space included a canal in the implant adapted to receive a screw head and allow passage of a screw into an adjacent vertebra. However, the device did not provide an opening along the length of one or more fixators that affix both vertebrae simultaneously.

In an eighth example a spinal implant device for fixing adjacent vertebra included a rod that spans the vertebrae, anchor seats and transpedicular screws that are assembled to affix adjacent vertebra. However, the device did not provide an opening along its length that would enable a second fixator to affix both vertebrae simultaneously within the disc space.

Thus there remains an unmet need for an improved implant for performing spinal surgical procedures (e.g., spinal fusion and/or fixations) that can securely fix the L5 vertebrae to the S1 sacrum within the disc space for patients exhibiting a wide range of anatomies. There further remains an unmet need for a surgical procedure for the installation or placement of such improved implant.

### SUMMARY OF INVENTION

The present invention provides for an improved implant, and surgical methods for installing same, that can securely fix the L5 vertebrae to the S1 sacrum within the disc space for patients exhibiting a wide range of anatomies.

The present invention has at least one advantage of providing practical and advantageous spinal implants and implantation systems, methods and tools for accessing the spinal vertebrae to insert spinal implants in various manners that overcome the above described disadvantages of posterior and anterior lateral approaches thereto and minimize surgical trauma to the patient.

The present invention for surgical methods. The tunnels created in the surgical method angle from the pelvis, diagonally into the outer bony area of the sacrum called the ala. From the alae the tunnels proceed in a straight line to the vertebral end plate of the sacrum and then enter the LS-S1 disc. Finally the tunnels exit the disc through the inferior end plate of the LS vertebral body and end within the LS vertebral body itself. One tunnel enters from each side of the body and each crosses the midline of the body within the LS-51 disc and ends in the contralateral side of the LS vertebral body. Through these tunnels the intervertebral disc can be removed, bone graft can be injected, and implants can be applied to stabilize the spinal segment.

The present invention allows both implants to be inserted across the L5-S1 disc by having one implant pass through the other implant by interlocking or interrelating the implants. This is accomplished by having a larger primary implant that has a slotted opening to allow the smaller diameter secondary implant to go through the larger implant.

In some aspects, the smaller diameter would compromise the degree of stabilization so it is further proposed that after passing through the larger implant fixation means would be deployed on the distal end of the secondary implant.

### BRIEF DESCRIPTION OF THE DRAWINGS

Examples illustrative of embodiments of the disclosure are described below with reference to figures attached hereto. In the figures, identical structures, elements or parts that appear in more than one figure are generally labeled with the same numeral in all the figures in which they appear. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings. The figures (Figs.) are listed below.

FIG. 1A illustrates a front profile of pathways drilled and created to provide access to the L5/S1 disc space.

FIG. 1B illustrates a side profile of pathways drilled and created to provide access to the L5/S1 disc space.

FIG. 2A illustrates a front profile of distraction and disc space preparation in the L5/S1 disc space.

FIG. 2B illustrates a side profile of distraction and disc space preparation in the L5/S1 disc space.

FIG. 3A illustrates a front profile of placement of distal fixation point of the primary fixator implant in the L5/S1 disc space.

FIG. 3B illustrates a side profile of placement of distal fixation point of the primary fixator implant in the L5/S1 disc space.

FIG. 4A illustrates a front profile of placement of the secondary fixator implant and impacting bone graft in the L5/S1 disc space.

FIG. 4B illustrates a side profile of placement of posterior screw implant and impacting bone graft in the L5/S1 disc space.

FIG. 5A illustrates a front profile of final assembly of primary and secondary fixator implants in the L5/S1 disc space.

FIG. 5B illustrates a side profile of final assembly of primary and secondary fixator implants in the L5/S1 disc space.

FIG. 6A illustrates a perspective and crossection view of the primary fixator implant interlocked with secondary fixator implant.

FIG. 6B illustrates a perspective view of the primary fixator implant interlocked with secondary fixator implant with the expansion member poised to enter the secondary fixator.

FIG. 7A illustrates a perspective view of the secondary fixator implant with fixation means in an expanded state (i.e. deployed).

FIG. 7B illustrates a cutaway view of the secondary fixator illustrating the internals of the secondary fixator means with the fixation means expanded.

FIG. 7C illustrates a cutaway view of the secondary fixator illustrating the internals of the secondary fixation means with the fixation means retracted internal to the secondary fixation means.

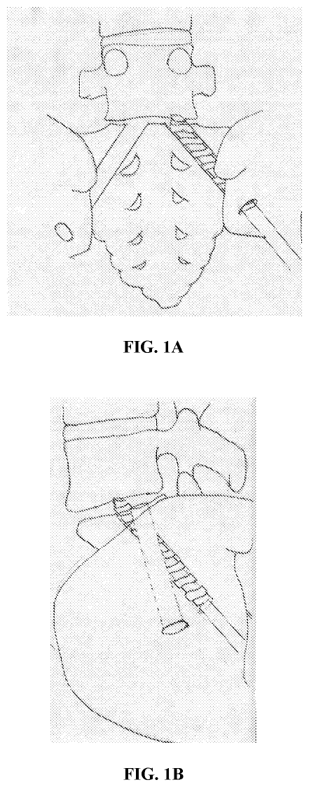

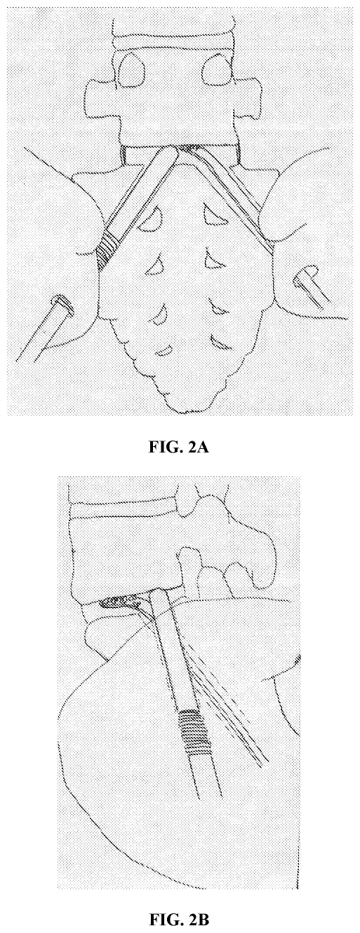

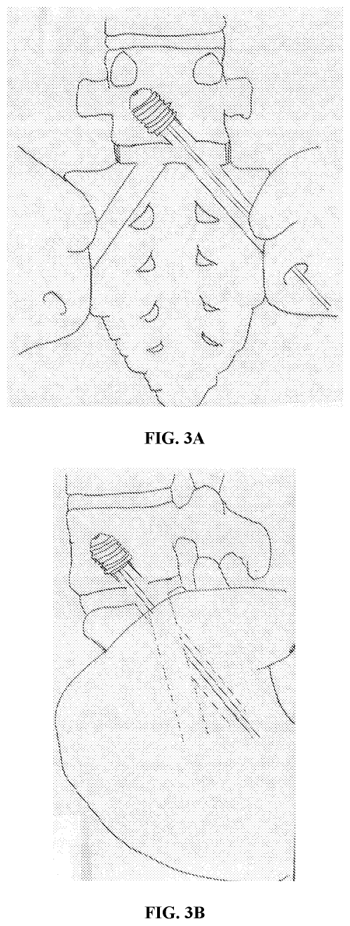

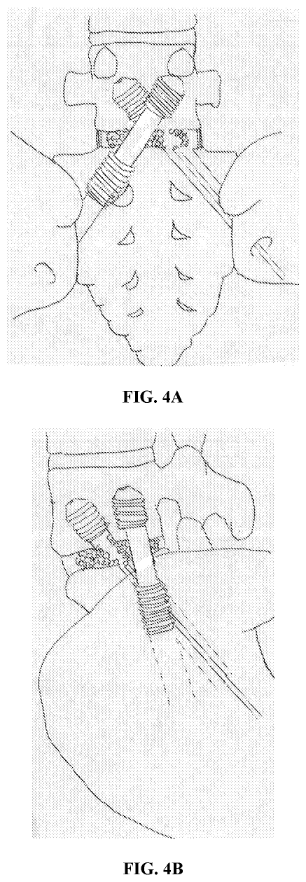

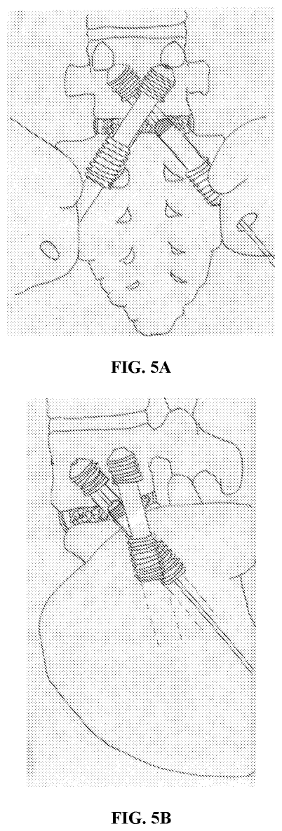

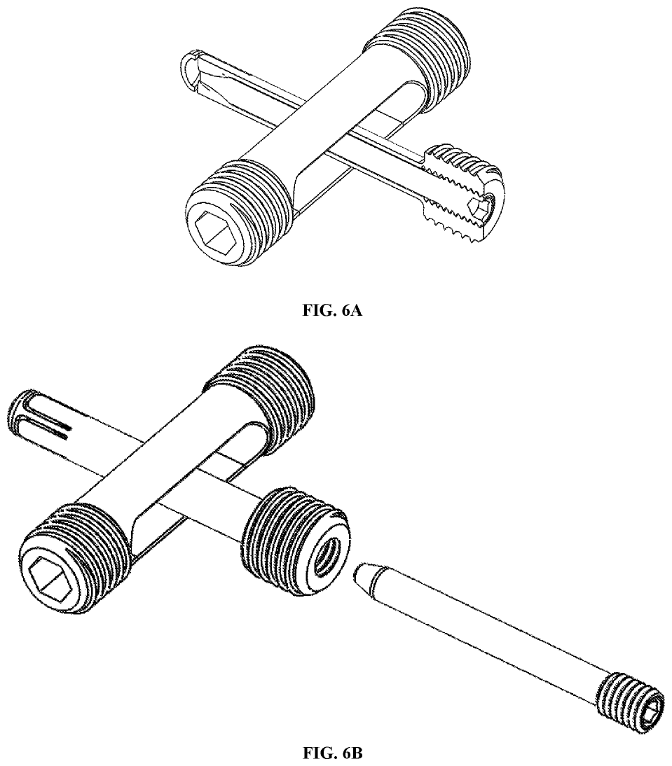

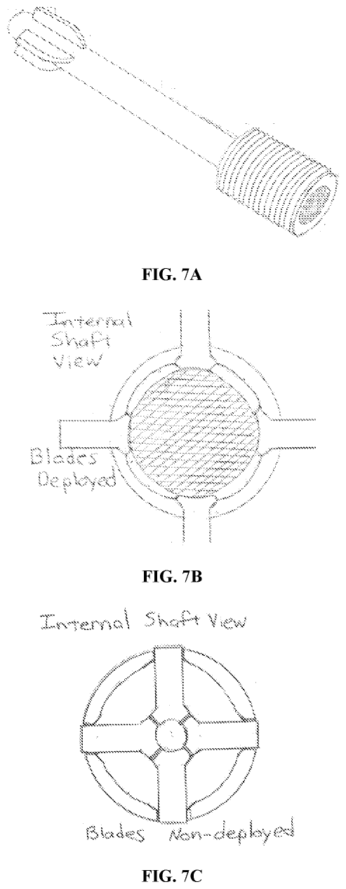

It should be clear that the description of the embodiments and attached Figures set forth in this specification serves only for a better understanding of the invention, without limiting its scope. It should also be clear that a person skilled in the art, after reading the present specification could make adjustments or amendments to the attached Figures and above described embodiments that would still be covered by the present invention.

### DETAILED DESCRIPTION OF INVENTION

This disclosure describes devices for performing spinal surgical procedures in which the L5 vertebra is affixed to the S1 sacrum of a human by a user. The present invention further provides devices, and methods for installing same, for performing spinal surgical procedures (e.g., spinal fusion and/or fixations) using one or more fixators to stabilize the L5 vertebrae and S1 sacrum.

It should be appreciated that on each side of the body there is a continuous bony corridor from the pelvis, through the sacrum, across the L5/S1 disc, and into the L5 vertebral body. Without being bound to any particular theory, these corridors can be traversed by a tunnel that would not damage or endanger any sensitive visceral or neurologic structures. The tunnels could be accessed by percutaneous incisions in the gluteal area. Thus the present invention provides for an improved implant, and surgical methods for installing same, that can securely fix the L5 vertebrae to the S1 sacrum within the disc space for patients exhibiting a wide range of anatomies and overcome the disadvantages of posterior and anterior lateral approaches thereto and minimize surgical trauma to the patient.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Unless defined otherwise, all technical and scientific terms used herein have the same meaning as commonly understood by one of ordinary skill in the art.

The phrase “ala”, as used herein, generally refers to the wing-like region of the sacrum in a Subject including, but not limited to, the area of bone extending laterally from the first sacral pedicle to the ilium.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

The term “fixation means” as used herein generally refers to one or more, or combinations, of material that when engaged or protruded fix a member to a desired location. For example, and without intending to limit the present disclosure, or any embodiment described herein, a fixation means may include one or more blades, talons, barbs, ribs, dowels, or other protrusions which extend from a secondary fixator to secure a secondary fixator in place during an implant installation.

The term “interconnected” as used herein generally refers to a plurality of pieces which may be joined, linked, inserted, traversing, and/or interrelated together. Without intending to limit the scope of the present invention, interconnected may be used to describe two or more pieces one of which may be inserted through, or intersect, but without physical connection, to the other, such that the two or more pieces may move independently and about each other, and may further have a range of motion limited as a result of the insertion, all without a physical connection linking the two. As a non-limiting example, and relative to the present invention, in at least one embodiment, a secondary fixation is inserted through a slot of a primary fixation, allowing independent movement of the primary fixation and secondary fixation along each axis of movement, but movement confined in relation to the width and height of the slot of the primary fixator.

The following description is of the best presently contemplated mode of carrying out the invention. This description is made for the purpose of illustrating the general principles of the invention and should not be taken in a limiting sense. The scope of the invention is best determined by reference to the appended claims.

### Implant Device

The present invention provides for a device for performing spinal surgical procedures (e.g., spinal fusion and/or fixations) of the L5 vertebrae and L5/S1 disc space. The inventive device has at least two or more fixators that interconnect along their axes. This interconnection may include a physical connection, or a relative non-physical connection or interrelation bounded by mechanical features of the fixators, such as slots limiting the amount of travel about one fixator in relation to the other.

Inventive devices include a primary fixator having two ends and an intermediate extent therebetween. The ends are at least one proximal end and at least one distal end. In some embodiments, the primary fixator has at least two sides (front facing side and rear facing side when viewing a fixator with its proximal and distal ends on the same horizontal plane). It is appreciated that with the selected shape of the primary fixator, a fixator may have any number of sides or a plurality of sides, and nothing herein is intended to limit the shape. Where fixators are cylindrical, a side in intended to mean a front facing and rear facing side from the perspective of the viewer and relative to the horizontal plane of the fixator. In at least one embodiment, the primary fixator may be threaded internally or externally on one or more of its ends. It is appreciated that for fixing an implant, typical implants are screwed to secure into place around the surrounding bone. However, nothing herein is intended to limit the method employed for fixing a primary fixator in place for surgical placement. In at least one embodiment, the intermediary extent of the primary fixator further includes a slot extending from the first side through to the second side of said primary fixator, the slot having a width and height for receiving at least one end of a secondary fixator. It is appreciated that nothing herein is intended to limit the width or height of the slot, except that the width and height should be selected to allow to securely fix the L5 vertebrae to the S1 sacrum within the disc space for patients exhibiting a wide range of anatomies and overcome the disadvantages of posterior and anterior lateral approaches thereto and minimize surgical trauma to the patient. It should be appreciated that width of the slot is intended to mean the horizontal distance of the slot created when viewing a side of the device having the slot with the distal end and proximal end on the same horizontal plane. It should be appreciated that height of the slot is intended to mean the horizontal distance of the slot created in relation to the width.

Inventive devices include further include a secondary fixator having at least one proximal end, at least one distal end, and an intermediate extent therebetween. In at least one embodiment, the secondary fixator may be threaded internally or externally on one or more of its ends. It is appreciated that for fixing an implant, typical implants are screwed to secure into place around the surrounding bone. However, nothing herein is intended to limit the method employed for fixing a primary fixator in place for surgical placement. In at least one embodiment, the secondary fixator is hollow or partially hollow. As a non-limiting example, in at least one embodiment the secondary fixator is hollow from the proximal end through a portion of the intermediate extent of the secondary fixator toward the distal end. In at least one embodiment, the secondary fixator may further include one or more slots or holes for allowing for the expansion or contraction of one or more fixation means, as further described later herein.

It is intended that certain embodiments of the present invention that the secondary fixator be able to move along all axis of movement to allow for the custom fit of the implant to a variety of anatomies. Accordingly, in at least one embodiment the primary fixator slot has a width at least sufficient to receive at least one end of a secondary fixator and allow movement of the secondary fixator along the width of said slot. While yet in at least one embodiment, the slot of the primary fixator has a height at least sufficient to receive at least one end of the secondary fixator and allow movement of the secondary fixator along the height of said slot. And yet, in at least one embodiment, the slot has both a width and height at least sufficient to receive at least one end of the secondary fixator and allow movement of the secondary fixator along the width and height of the slot.

It is appreciated that while some aspects of the inventive device may be fixed through having threads at either or both of the proximal or distal end, some embodiments may use alternative fixing means for fixing one or more ends of a primary or secondary fixator in place. In at least one embodiment, one end of the secondary fixator includes one or more fixation means which are configured to expand or retract from said secondary fixator. In such embodiments, the fixator can be any fixation means known in the art, but include, without limit, one or more blades, dowels, fins, talons, barbs, ribs, knurls, bumps, grooves, or other protrusion type or shape known in the art. Use of the fixation means allows for the insertion of a secondary fixator through a slotted primary fixator, while allowing for the fixation of the distal end of the secondary fixator in a desired location when placing of an implant and allows for the variability in the patients anatomy to dictate the size of the fixation means.

As discussed above, certain embodiments of the present invention may include one or more hollow portions of a secondary fixator. Such hollow portion may be configured for receiving one or more expansion shaft. Such expansion shafts are intended to cause a portion of the secondary means to expand, primarily for fixation into one or more locations in the human anatomy. Such expansion increases the fixation strength of the fixator to the bone due to its increased surface area as compared to a smaller diameter screw thread. Accordingly, in some embodiments which utilize an expanding (and/or contracting) fixation means, the expansion shaft is intended to engage with such fixation means to cause such fixation means to expand from and/or contract into the secondary fixator. Inventive expansion shafts have at least one proximal end, at least one distal end, and an intermediate extent therebetween. In some embodiments, the expansion shaft is threaded on at least one proximal end or at least one distal end to be threaded into the secondary fixation. In such embodiments, the secondary fixation has internal threads for receiving an externally threaded expansion shaft. In at least one embodiment, the expansion shaft is configured on at least one proximal end or at least one distal end to engage one or more fixation means, where upon tightening the expansion shaft into the secondary fixator, the one or more fixation means extend from the slots and hollow portion of the secondary fixator. In at least one embodiment, whereupon loosening the expansion shaft in relation to the secondary fixator, the one or more fixation means retract into the hollow portion of said secondary fixator. In some embodiments, the width and/or height of said secondary fixation measured at the said one or more fixation means of the secondary fixation, are larger than the width and/or height of slot of the primary fixator, thus mechanically limiting the relationship of the primary fixator and the secondary fixator about the length of the primary fixator between the proximal and distal ends. Likewise, it is intended that when retracted the width and/or height of said secondary fixation measured at the said one or more fixation means of the secondary fixation, are smaller than the width and/or height of slot of the primary fixator.

Embodiments of the invention may further include multiple lengths and diameters of the primary and/or secondary to accommodate a broad diversity of patient anatomies.

It is appreciated that the materials selected for the construction of any of the elements of the implant device may include such material known in the art and typically used in the construction of surgical implants, including, without limit, stainless steel, titanium, PEEK, ceramics, durable plastics, polymers, porous structures and coatings or combinations thereof.

### Surgical Method

It should be appreciated that as part of this invention, that a bony pathway on each side of the spine, leading from the iliac crest of the pelvis directly through the pedicle of the first sacral vertebra (“S1”) and then to the vertebral body of the fifth lumbar vertebra (“L5”) on each side of the spine, provides an optimum pathway for placement of surgical instrumentation to effect fixation and fusion and/or fixation of the lumbosacral joint. Thus allowing for the optimal placement of one or more inventive devices described herein.

To take advantage, the present invention further includes a surgical method for the installation of a spinal fusion and/or fixation device (as may be described in any and all embodiments described herein) in a human for the L5 vertebrae, L5/S1 disc space and S1 sacrum. Inventive surgical methods include creating at least one pathway to provide access to the L5/S1 disc space, preparing the pathway in order to receive at least one of a primary fixator or a secondary fixator, installing a primary fixator in the L5 vertebrae and S1 sacrum, and installing a secondary fixator that interconnects/intersects through one or more slot in the primary fixator. In embodiments where a secondary fixator is used which includes one or more expanding or contracting fixation means, the method further includes expanding one or more fixation means in place. As described herein, in some embodiments expansion of one or more fixation means may be accomplished by the tightening of one or more expansion shaft in the secondary fixator.

In at least one embodiment, the surgical method may further include cannulation and bone grafting. It is appreciated that the cannulation of the primary and/or fixations is primarily for allowing a preliminary guide wire to ensure the secondary fixation engages with the slot of the primary implant. In at least one embodiment, the bone graft (and disc removal) will occur prior to the placement of the implants, through the pre-drilled bony tunnels.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A device for performing spinal surgical procedures (e.g., spinal fusion and/or fixation) of the L5 vertebrae and L5/S1 disc space having at least two or more fixators that interconnect along their axes, the device comprising:
a primary fixator having two ends and an intermediate extent therebetween, wherein said primary fixator has at least two sides and wherein said ends include at least one proximal end and at least one distal end; and
a secondary fixator having at least one proximal end, at least one distal end, and an intermediate extent therebetween.

2. The device of claim 1 wherein said at least one of said proximal end or distal end of said primary fixator includes screw threads.

3. The device of claim 1 wherein said at least one of said proximal end or distal end of said secondary fixator includes screw threads.

4. The device of claim 1 wherein said intermediary extent of said primary fixator further comprises of a slot extending from the first side to the second side of said primary fixator, wherein said slot has a width and height at least for receiving at least one end of said secondary fixator.

5. The device of claim 4 wherein the slot has a width at least sufficient to receive at least one end of said secondary fixator and allow movement of the secondary fixator along the width of said slot.

6. The device of claim 4 wherein the slot has a height at least sufficient to receive at least one end of said secondary fixator and allow movement of the secondary fixator along the height of said slot.

7. The device of claim 4 wherein the slot has a width and height at least sufficient to receive at least one end of said secondary fixator and allow movement of the secondary fixator along the width and height of said slot.

8. The device of claim 1 wherein said distal end of said secondary fixator includes one or more fixation means which are configured to expand or retract from said secondary fixator.

9. The device of claim 8 wherein said secondary fixator is at least partially hollow.

10. The device of claim 9 wherein said hollow secondary fixator is hollow from the proximal end through a portion of the fixator toward the distal end.

11. The device of claim 9 further comprising one or more expansion shaft wherein said hollow secondary fixator is configured to receive said one or more expansion shaft.

12. The device of claim 11 wherein said expansion shaft has at least one proximal end, at least one distal end, and an intermediate extent therebetween.

13. The device of claim 12 wherein said expansion shaft is threaded on at least one proximal end or at least one distal end to be threaded into said secondary fixation.

14. The device of claim 13 wherein said expansion shaft is configured on at least one proximal end or at least one distal end to engage said one or more fixation means, wherein upon tightening said expansion shaft into said secondary fixator, said one or more fixation means extend from said hollow portion of said secondary fixator.

15. The device of claim 14 wherein said width or height of said secondary fixation measured at the said one or more fixation means of the secondary fixation, are larger than the width and height of slot of the primary fixator.

16. The device of claim 13 wherein said expansion shaft is configured on at least one proximal end or at least one distal end to engage said one or more fixation means, wherein upon loosening said expansion shaft out of said secondary fixator, said one or more fixation means retract into said hollow portion of said secondary.

17. The device of claim 16 wherein said width or height of said secondary fixation measured at the said one or more fixation means of the secondary fixation, are smaller than the width and height of slot of the primary fixation when said one or more fixation means are retracted.

18. A surgical method for the installation of a fixation device in a human to stabilize the L5 vertebrae to the S1 sacrum having at least two or more fixators that intersect along their axes, wherein said device include a primary fixator having two ends and an intermediate extent therebetween, wherein said primary fixator has at least two sides and wherein said ends include at least one proximal end and at least one distal end, and a secondary fixator having at least one proximal end, at least one distal end, and an intermediate extent therebetween, the method comprising:
creating at least one pathway to provide access to the L5/S1 disc space;
preparing the pathway in order to receive at least one of a primary fixator or a secondary fixator;
installing a primary fixator in the L5 vertebrae and S1 sacrum; and
installing a secondary fixator that intersects with the primary fixator.

19. The method of claim 18 wherein said secondary fixator is hollow and is configured for receiving one or more expansion shaft, wherein said method further comprises installing said expansion shaft in said secondary fixator.

20. The method of claim 19 wherein said hollow secondary fixator includes one or more fixation means configured to expand upon engagement from an expansion shaft, wherein said method further comprises tightening said expansion shaft in said secondary fixator to expand said one or more fixation means of said secondary fixator.
